(19) United States
(12) Patent Application Publication
(10) Pub. No.: US 2022/0023454 A1
(43) Pub. Date: Jan. 27, 2022

(54) PHOTODYNAMIC METHOD TO DECONTAMINATE SURFACES

(71) Applicants: Hamada Aboubakr, St. Paul, MN (US); Sagar Goyal, Roseville, MN (US)

(72) Inventors: Hamada Aboubakr, St. Paul, MN (US); Sagar Goyal, Roseville, MN (US)

(21) Appl. No.: 17/382,869

(22) Filed: Jul. 22, 2021

Related U.S. Application Data
(60) Provisional application No. 63/055,220, filed on Jul. 22, 2020.

(51) Int. Cl. A61L 2/08; A01N 43/16; A01N 43/90; A01N 59/20; A23B 4/015; A23B 7/015

(52) U.S. Cl. CPC A61L 2/088; A01N 43/16; A01N 43/90; A01P 1/00; A23B 4/015; A23B 7/015; A01N 59/20

(57) ABSTRACT

A method to decontaminant surfaces including the surface of food and aqueous solutions is provided.

### CROSS-REFERENCE TO RELATED APPLICATIONS

This application claims the benefit of the filing date of U.S. application No. 63/055,220, filed on Jul. 22, 2020, the disclosure of which is incorporated by reference herein.

### BACKGROUND

Human noroviruses (HuNoVs) are responsible for 125,000,000 foodborne illnesses and 35,000 foodborne deaths. The annual global economic burden of HuNoV is estimated at $64.5 billion in direct health system costs and societal costs (Bartsch et al., 2016). HuNoVs are responsible for 48% of foodborne outbreaks that are linked to a single known pathogen. The annual economic cost of foodborne illnesses is $77 billion in the U.S. alone (Scharff, 2012) (FIG. 1).

Epidemiological studies reported that fresh produce, berries and other fruits, and bivalve mollusks and shellfish are the most implicated food commodities in HuNoV outbreaks in the US. These foods can be contaminated following the use of virus-contaminated waters in shellfish farms or in pre/post-harvest processes of fresh produce. To mitigate this risk, many non-thermal intervention processes have been studied or are in use in industry, such as chlorination, ozone treatment, and UV. However, each of these processes has its own drawbacks.

### SUMMARY

The disclosure provides a method for the decontamination of food, water, and surfaces from viral contamination using photodynamic technology. As described herein, methods and apparatus are provided for preventing or inhibiting, e.g., reducing or inactivating, the viability of viruses on surfaces, e.g., food surfaces or inanimate surfaces used for food preparation, or in solution, e.g., water used to wash food, water used to store and/or depurate shellfish, or waste water, with light and a photosensitizer, e.g., a biocompatible photosensitizer. In one embodiment, an apparatus utilizes one or more blue light emitting diodes (LED) to irradiate a surface or a solution that is contacted with a photosensitizer in order to inhibit or reduce the viability or infectivity of virus on the surface or in the solution. Light emission and photosensitizer contact with a surface or solution may be applied periodically.

Thus, the method utilizes light, such as blue light, and a photosensitizer to inhibit viral viability or inactivate infectivity of viruses on surfaces or in solutions. In one embodiment, the light for exposure includes, but is not limited to, blue light at a wavelength within a range of 450 nm to 480 nm (e.g., 455 nm to 475 nm). In one embodiment, the surface or solution having the photosensitizer is exposed to the light for 1, 2, 3, 5, 7, 10, 15, or 20 minutes or more. In one embodiment, the distance between the light source and the surface or food source is at least 0.5, 1, 2, 3, 4, 5, 10, 15 or 20 cm.

In one embodiment, a method to decrease microbial load, e.g., viral load on a surface, food product or in an aqueous solution is provided. The method includes providing a surface, food product or an aqueous solution suspected of having virus or bacterium; and exposing the surface, food product or solution to an amount of a photosensitizer and an amount of blue light for a period of time effective to decrease viral or bacterial load on the surface or the food product or in the aqueous solution. In one embodiment, the surface is exposed to the photosensitizer and the blue light. In one embodiment, the aqueous solution is exposed to the photosensitizer and the blue light. In one embodiment, the photosensitizer is a fluorescein. In one embodiment, the fluorescein is halogenated. In one embodiment, the photosensitizer is halogenated. In one embodiment, the photosensitizer has 1 to 4 Cl, Fl, Br or I. In one embodiment, the photosensitizer has 1 to 4 Cl and 1 to 4 Br. In one embodiment, the photosensitizer has 1 to 4 Cl and 1 to 4 I. In one embodiment, the photosensitizer is a red dye. In one embodiment, the photosensitizer is Rose Bengal, Phloxine B, chlorophyllin, or a salt thereof. In one embodiment, the light has a wavelength of about 450 nm to 490 nm or 460 nm to 475 nm or 460 nm, or any wavelength of visible light. In one embodiment, the light has a wavelength of about 460 nm to 480 nm. In one embodiment, the light has a wavelength of about 450 nm to 470 nm. In one embodiment, the virus is an enveloped virus. In one embodiment, the virus is a non-enveloped virus. In one embodiment, the virus is a single or doubled stranded DNA virus. In one embodiment, the virus is a single or doubled stranded RNA virus. In one embodiment, the virus is a parvovirus, poxvirus, herpesvirus, hepatitis virus, enterovirus, norovirus, or coronavirus. In one embodiment, the viral or bacterial load is decreased by at least 2 logs. In one embodiment, the viral or bacterial load is decreased by at least 6 logs. In one embodiment, the food product is exposed to the light and the photosensitizer. In one embodiment. the food product is produce or fruit. In one embodiment, the food product is shellfish. In one embodiment, the surface is a steel, granite, marble, wood, rubber, ceramic, plastic, fabric, or glass surface. In one embodiment, the aqueous solution has a pH from 5 to 9. In one embodiment, the aqueous solution has a pH from 6 to 8.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1. Burdens of NoVs on US economy and public health.

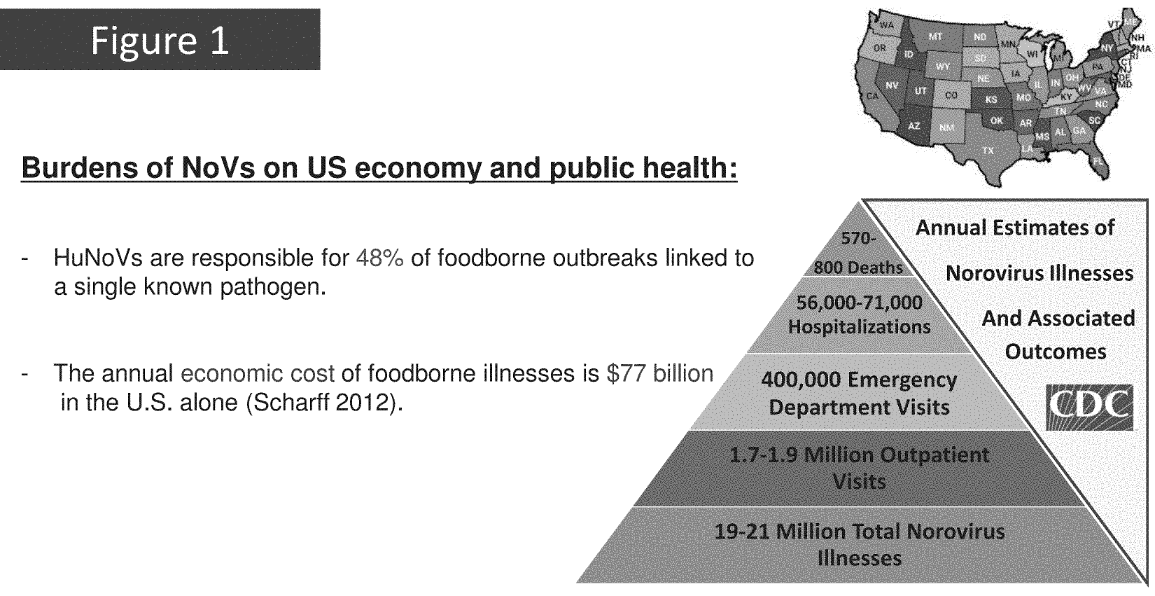

FIG. 2. Proportion of implicated single food commodities among 101 GII.4 and 72 non-GII.4 foodborne norovirus outbreaks reported to the National Outbreak Reporting System and CaliciNet, United States, August 2009-July 2015 (Marsh et al., 2018)

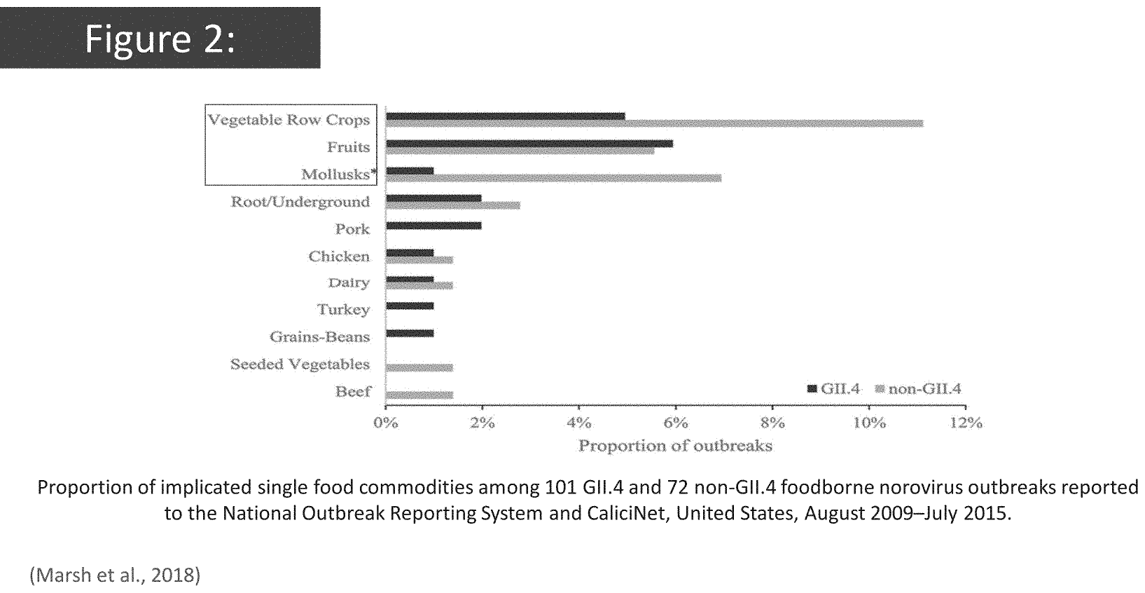

FIG. 3. Photodynamic treatment (PDT).

FIG. 4. Exemplary photosensitizers (PSs).

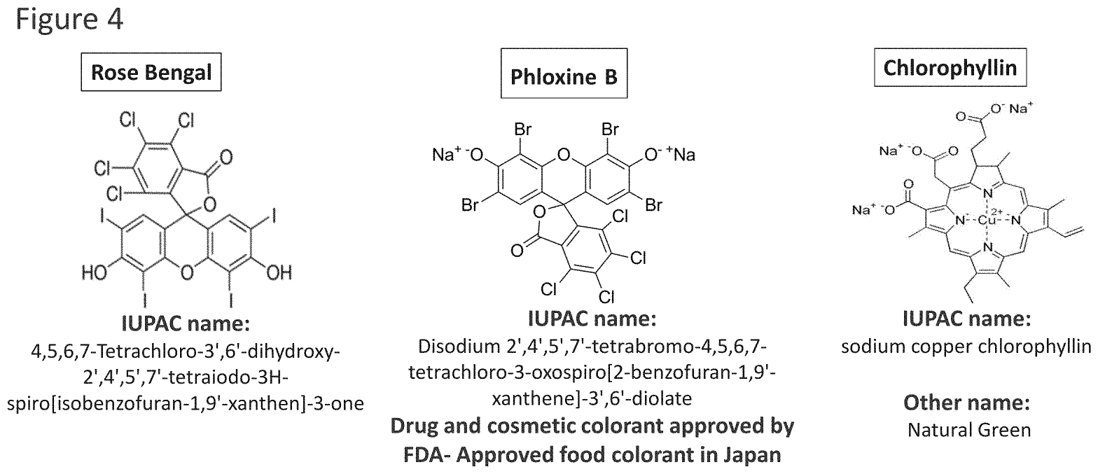

FIG. 5. Light sources tested.

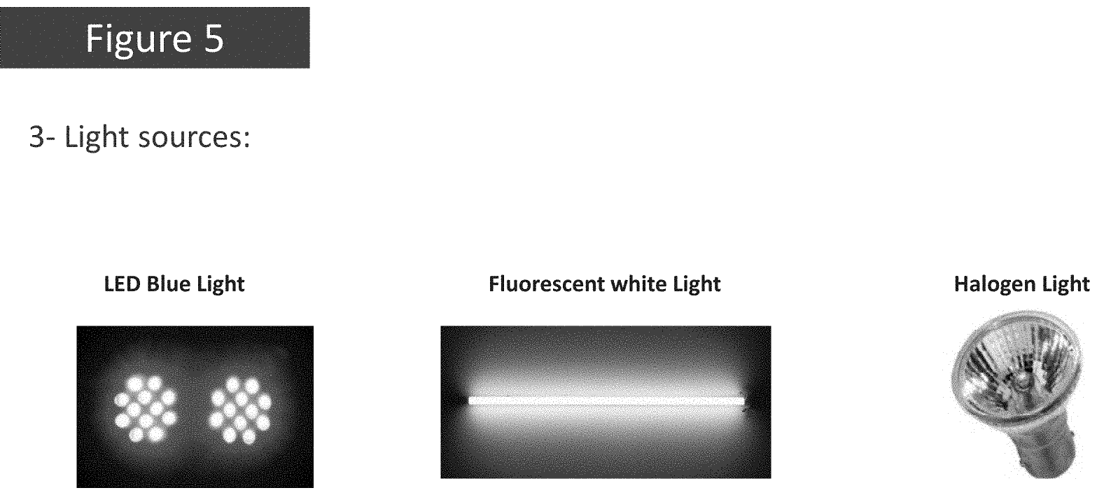

FIG. 6. Exemplary blue light emitting device.

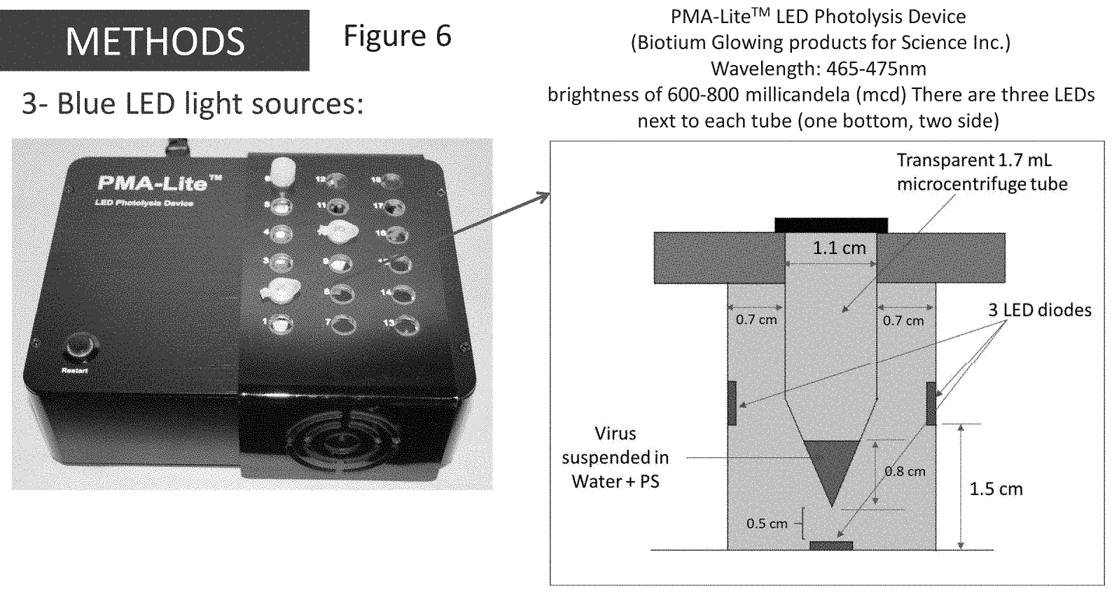

FIG. 7. Possible mode of anti-viral activity for PDT/PS.

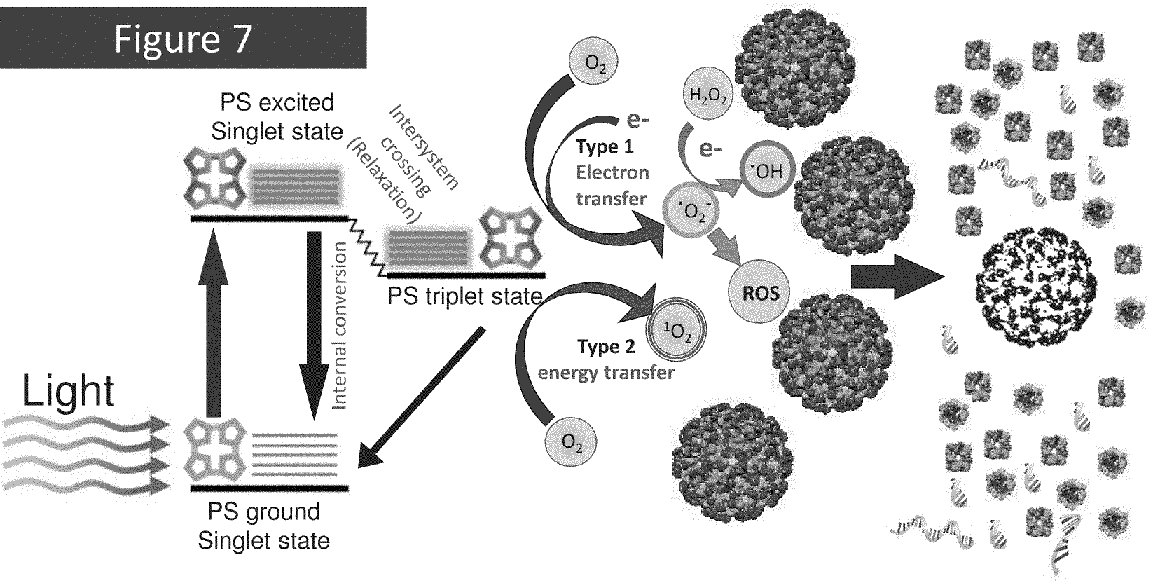

FIG. 8. Results for RB and FCV exposed to three different types of light with three different wavelengths for 10 minutes.

FIG. 9. Results for PSs and FCV exposed to blue light for 10 minutes

FIG. 10. Results for three different concentrations of RB and PB, and FCV, exposed for three different periods of time to blue LED light.

FIG. 11. Results for three different concentrations of chlorophyllin, and FCV, exposed for three different periods of time to blue LED light.

FIG. 12. Results for three different concentrations of RB and FCV exposed for three different periods of time to blue light.

FIG. 13. Results for three different concentrations of RB and Ph-B, and TuV, exposed for three different periods of time to blue light.

FIG. 14. Decontamination of steel surfaces and the effect of exposure distance using RB and light source A.

FIG. 15. Decontamination of steel surfaces and the effect of exposure distance using PB and light source A.

FIG. 16. Decontamination of steel surfaces and the effect of exposure distance using RB and light source B.

FIG. 17. Decontamination of steel surfaces and the effect of exposure distance using Ph-B and light source B.

FIG. 18. RNase-coupled RT-qPCR for studying capsid integrity.

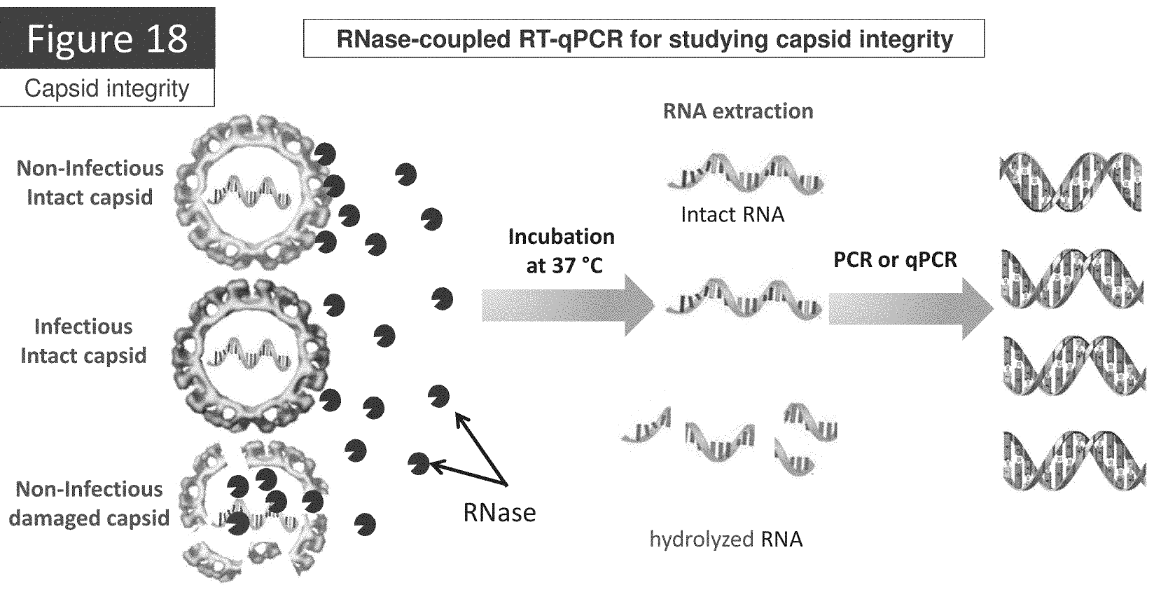

FIG. 19. Results for RNase-coupled RT-qPCR for studying capsid integrity.

FIG. 20. TEM image of TuV (50 μM) after exposure to blue LED light for 10 minutes in the presence of RB or Ph-B.

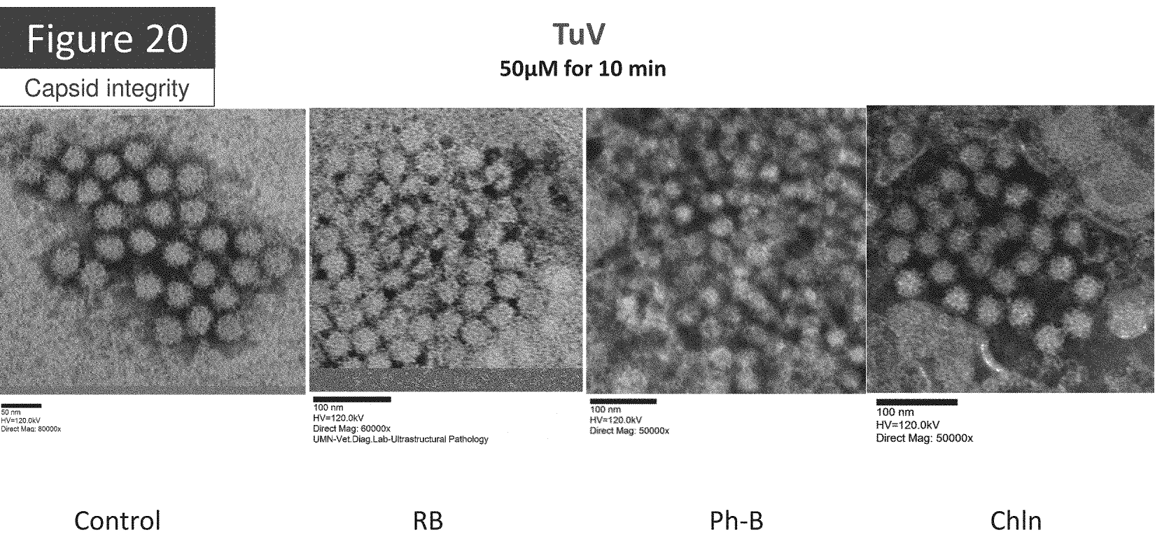

FIG. 21. RT-PCR results for FCV genome after exposure to blue LED light in the presence of RB and Ph-B for different periods of time.

FIG. 22. RT-PCR results for TuV genome after exposure to blue LED light in the presence of and RB and PB.

FIG. 23. Exemplary system for decontamination of steel surfaces and romaine lettuce leaves samples having transmissible gastroenteritis virus (TGEV), a surrogate of coronaviruses, with light source B.

FIG. 24. Decontamination of steel surfaces and romaine lettuce leaves from TGEV as a surrogate of coronaviruses with light source B.

FIG. 25. Data for decontamination of steel surfaces and romaine lettuce leaves from TGEV as a surrogate of coronaviruses with light source B.

### DETAILED DESCRIPTION

In the following detailed description, reference is made to the accompanying drawings which form a part hereof, and in which is shown by way of illustration specific embodiments in which the invention may be practiced. These embodiments are described in sufficient detail to enable those skilled in the art to practice the invention, and it is to be understood that the embodiments may be combined, or that other embodiments may be utilized, and that structural, logical and electrical changes may be made without departing from the spirit and scope of the present invention. References to “an”, “one”, or “various” embodiments in this disclosure are not necessarily to the same embodiment, and such references contemplate more than one embodiment. The following detailed description provides examples, and the scope of the present invention is defined by the appended claims and their legal equivalents.

As used herein, a light source which “substantially” emits a particular band or wavelength(s) of light is a light source in which more than 50%, preferably more than 60%, and more preferably more than 80%, of the band or wavelength(s) that are emitted are of the specified band or wavelength(s). For instance, a light source which emits substantially red light emits more than 50%, more than 60%, or more than 80%, of the total light in the red portion of the spectrum. The blue portion of the spectrum is defined herein as wavelengths longer than about 450 nm and less than about 485 nm, a frequency of about 620 THz to 680 THz, and or a photon energy of about 2.64 eV to 2.75 eV.

### Decontamination

Currently, there is no available commercial non-thermal technology to decontaminate food, water, shellfish and food processing surfaces, e.g., contaminated with viruses such as norovirus, e.g., human noroviorus (HuNoV), or other enteric viruses, without affecting its quality and nutritive value. The only way the food industry and food service businesses mitigate this problem is to follow strict food processing hygiene and strict personal hygiene for the food handlers. Other nonthermal technologies have been developed at research scale such as cold plasma technology, high pressure processing technology, and pulsed electric field but all have limitations in terms of cost and/or impact on the quality of the treated food.

As reported by the CDC, HuNoV is the leading cause of foodborne gastroenteritis outbreaks in the US. It leads to 21 million illnesses in the US every year including 1.7-1.9 million outpatient visits, 400,000 emergency room visits, 56,000-71,000 hospitalizations, and 570-800 deaths. Fresh produce and leafy vegetables, berries, shellfish, and food-contact surfaces in restaurants and cruise ships are the most implicated in HuNoV outbreaks. Because these foods are usually consumed fresh, thermal treatments cannot be used to decontaminate them from viral contaminants as it affects its organoleptic and nutritive qualities. Therefore, there is a real need to develop inexpensive non-thermal processes for decontamination of these types of foods from viral contaminants without affecting their quality.

As described herein, photodynamic treatment (PDT) using photosensitizers (PS) was found to be effective in decontaminating food, water, and surfaces from viral pathogens, e.g., norovirus such as HuNoV, hepatitis A virus (HAV) or coronavirus, such as SARS-CoV-2, employing in one embodiment an edible PS and a low energy consuming and inexpensive light source. In one embodiment, Phloxine B (an edible food and cosmetic colorant) was employed as a photosensitizer and a blue LED light source was used to emit certain wavelengths of light. In one embodiment, the use of blue light and a PS may result in a reduction in virus (virus load as measured by any method) of more than 99.99% of HuNoV and SARS-CoV-2 surrogates within 5 minutes or 1 minute, respectively, of exposure.

Thus, PDT and PS, e.g., a biocompatible PS, may be employed in various applications. For example, a small home use scale device may be employed for washing and decontaminating green leafy produce and berries that are eaten fresh, thus eliminating extensive washing under running water or even soaking in water mixed with vinegar which is unable to decontaminate foods from viral contaminants.

In one embodiment, PDT and PS may be employed in fruit and vegetable washing machines in a food processing line and/or in postharvest stations.

In one embodiment, PDT and PS may be employed in the shellfish industry by integrating it with the depuration process to make it more efficient by not only eliminating the virus particles from the shellfish stomach but also by inactivating the virus particles.

In one embodiment, PDT and PS may be employed in a hand sanitizing device, e.g., in the restaurant or healthcare fields, that is not dependent on alcohol or bleach, which have side effects on the health of hands and its skin.

In one embodiment, PDT and PS may be employed with a surface sanitizing device that can be used in restaurants to decontaminate tables, plates, cutting boards, and/or utensils. In one embodiment, PDT and PS may be employed with a surface sanitizing device that can be used in nursing homes and hospitals to decontaminate tables, plates, cutting boards and/or utensils

In one embodiment, PDT and PS may be employed in dish washing machines to ensure the inactivation of viruses.

### Exemplary Photosensitizers for Use in the Methods

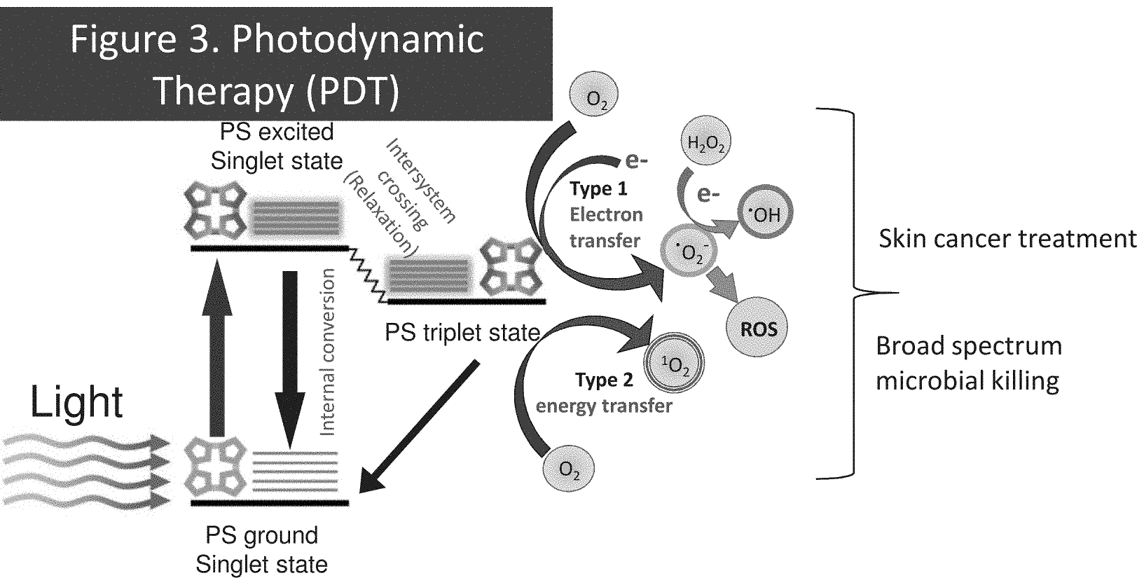

FIG. 3 is an illustration of photodynamic treatment (PDT) involving the use of visible light to excite photosensitizers (PS). In the presence of molecular ground (triplet)-state oxygen (³O₂), the excited-state PS transfer energy or electrons to oxygen to producing reactive oxygen species (ROS) that are able to kill cells or pathogens.

The inactivation of HuNoV surrogates (e.g., feline calicivirus (FCV) and Tulane virus (TuV)) was studied, by exposure to light in the presence of photosensitizer dyes in water, FCV and TuV were mixed with different concentrations (10, 25, and 50 μM) of three different PS (Rose Bengal (RB), Phloxine B (Ph-B) and chlorophyllin) and then exposed to light for a period of time, e.g., 1, 3, 5, or 10 minutes. While not being limited to a mode of action, capsid integrity and/or genome integrity may be impacted by the exposure of viruses to light in the presence of the PS.

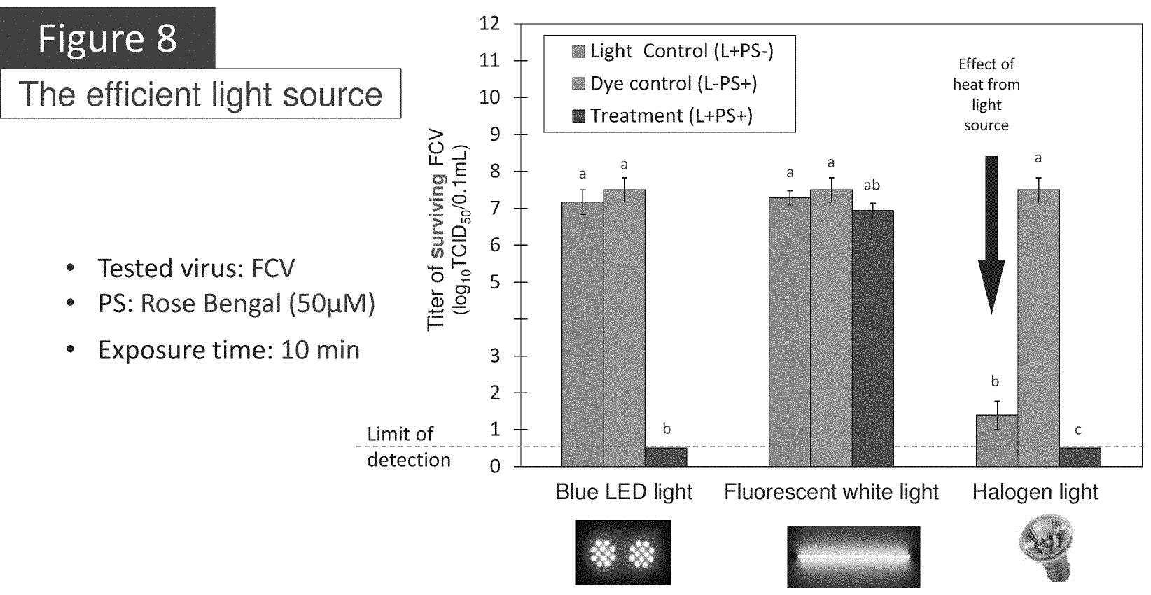

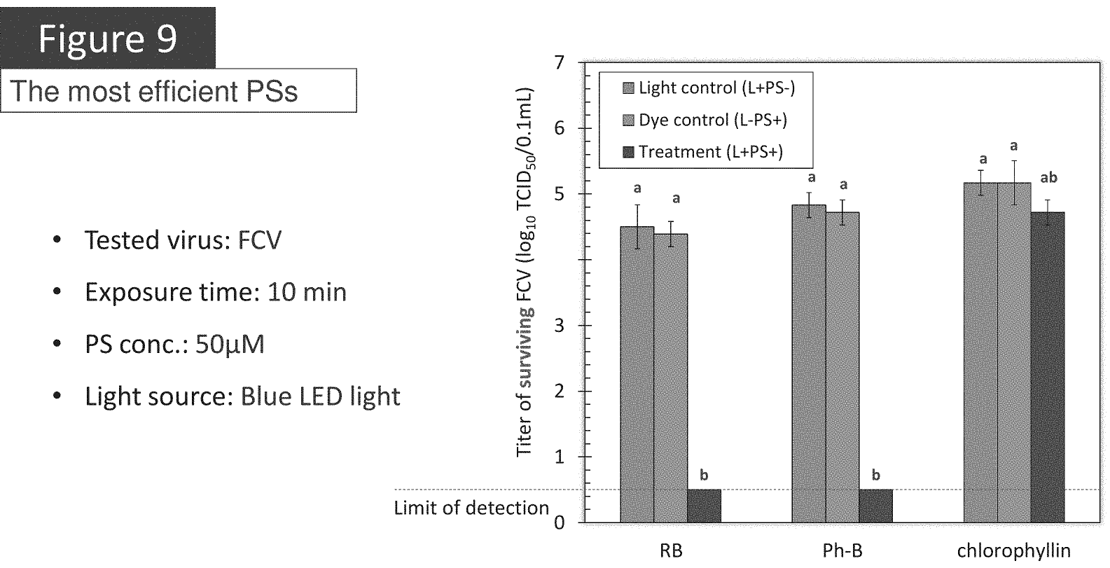

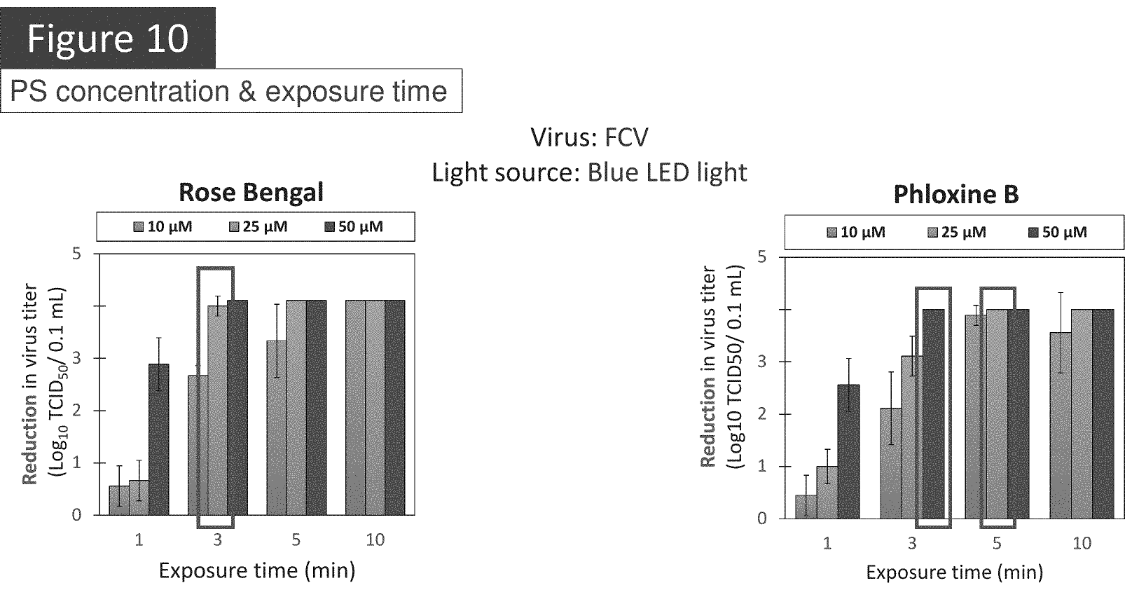

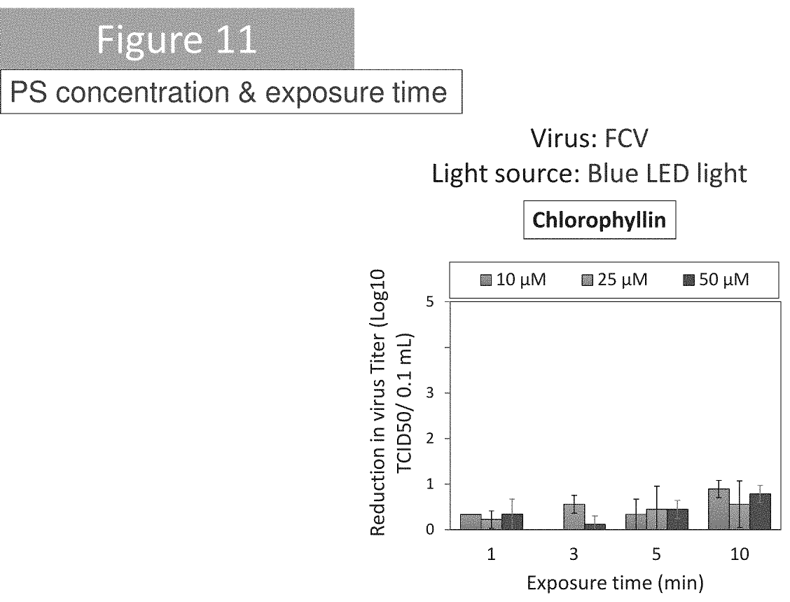

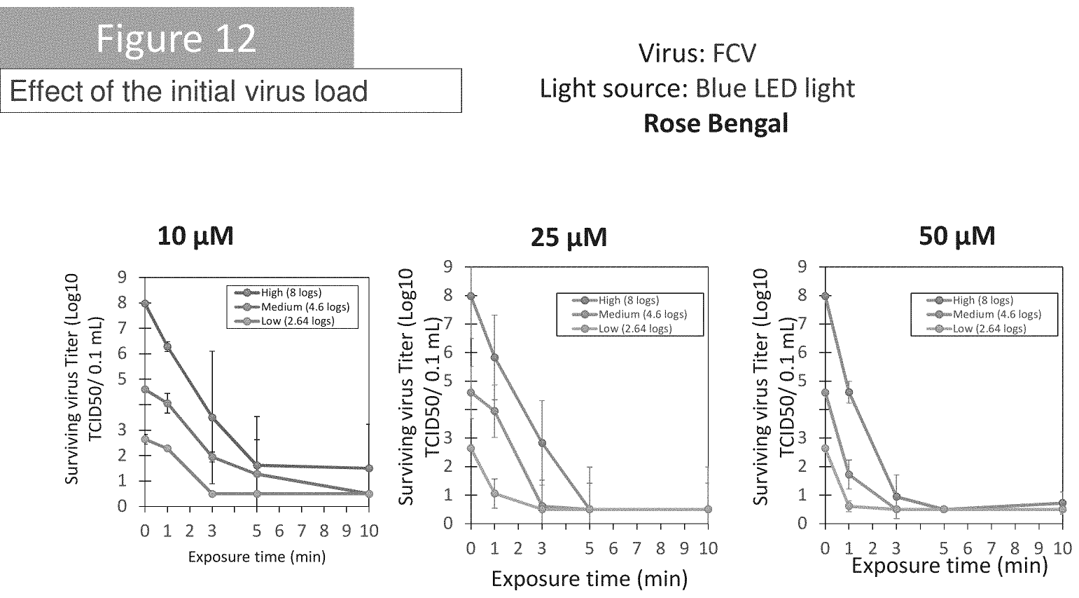

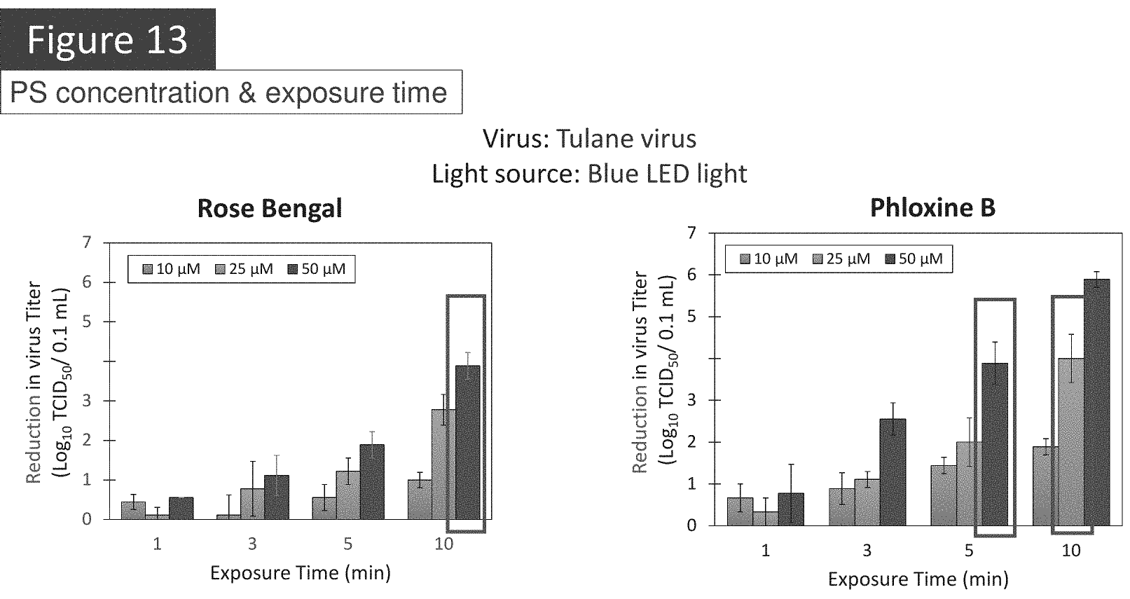

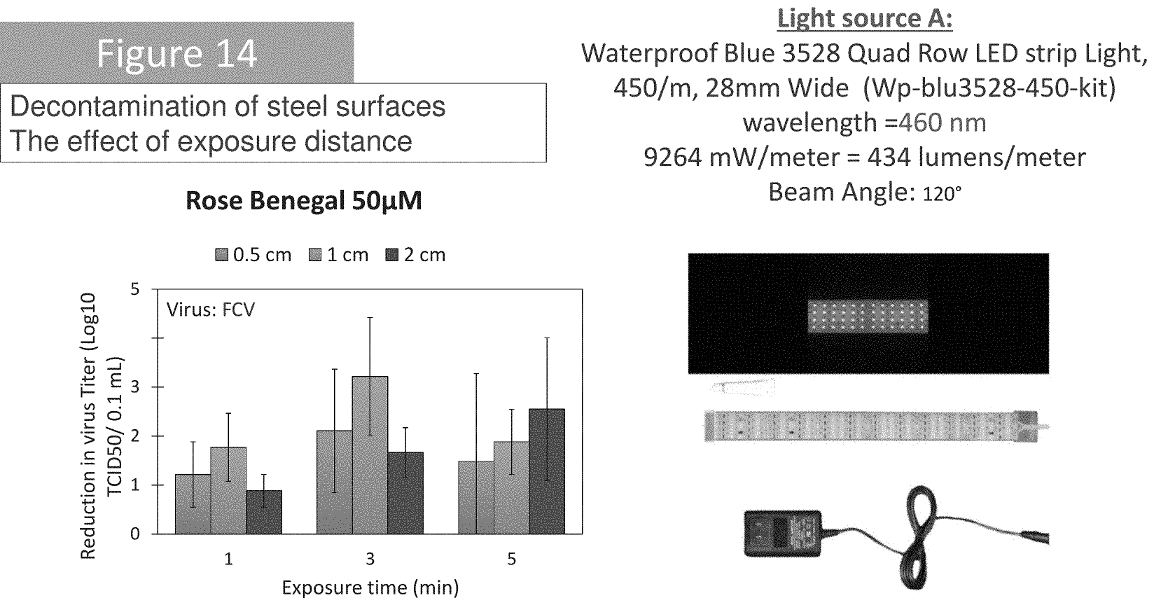

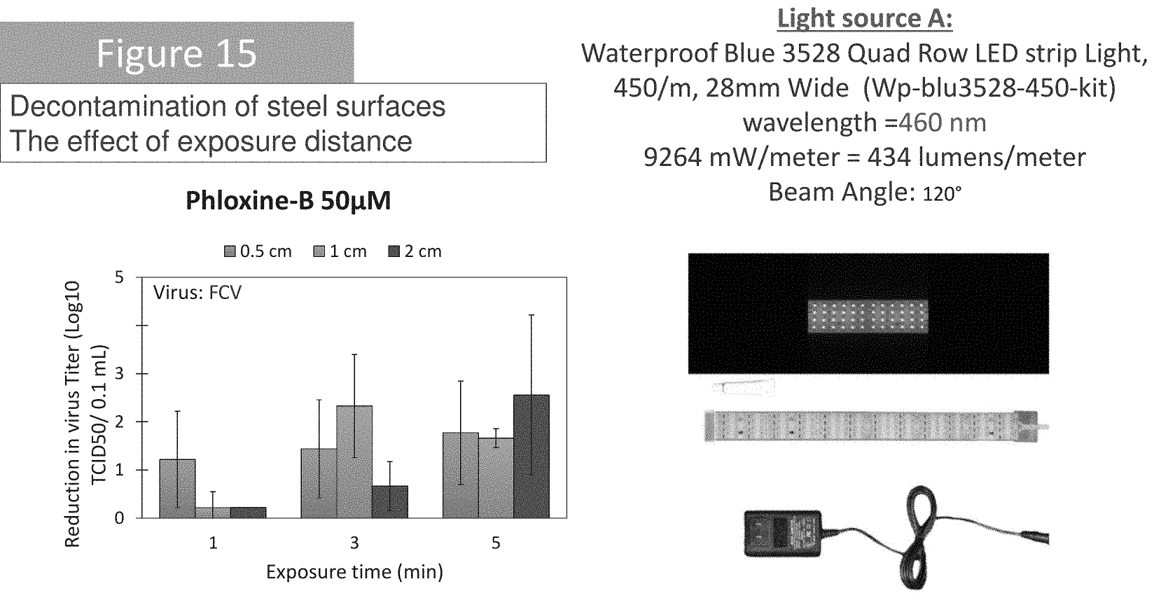

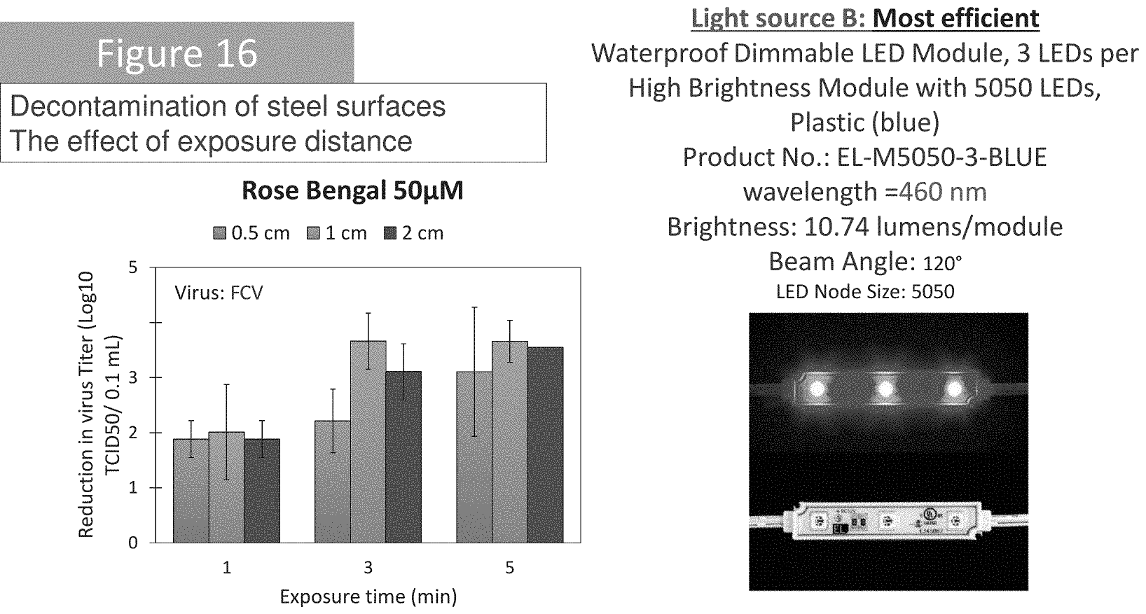

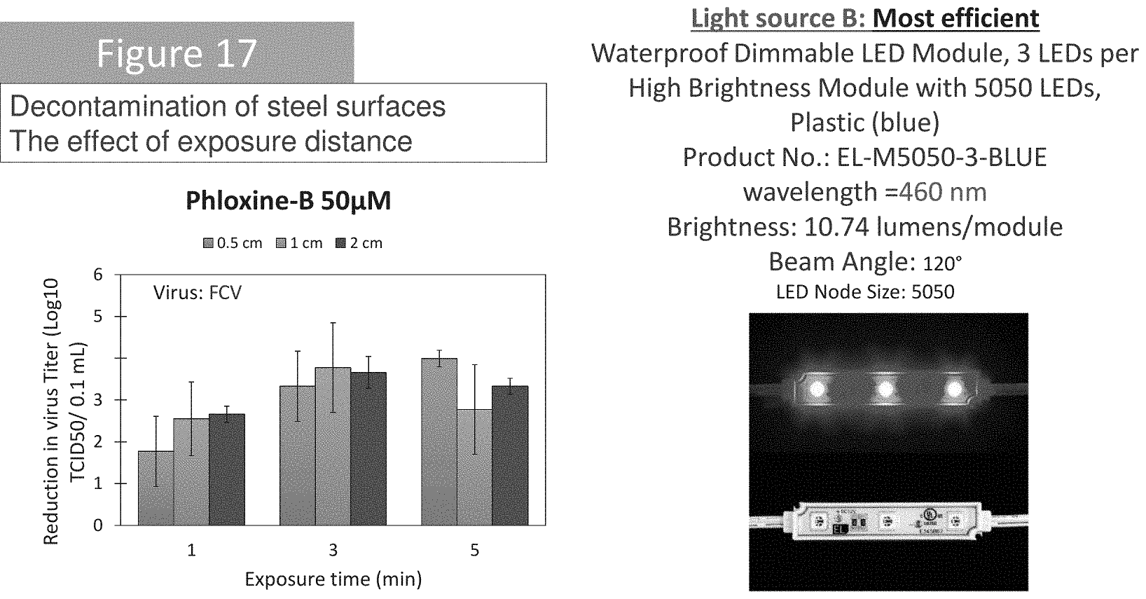

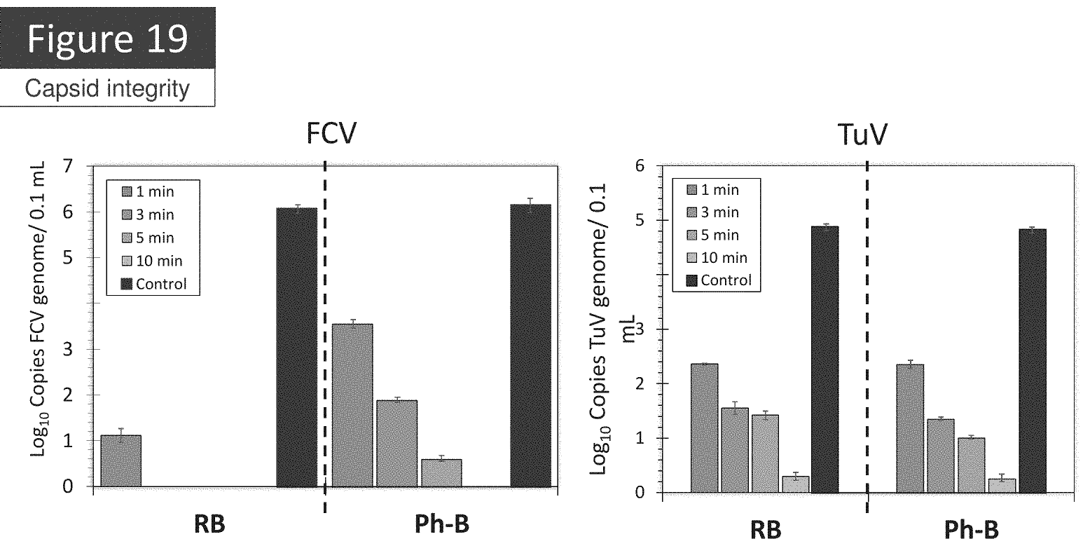

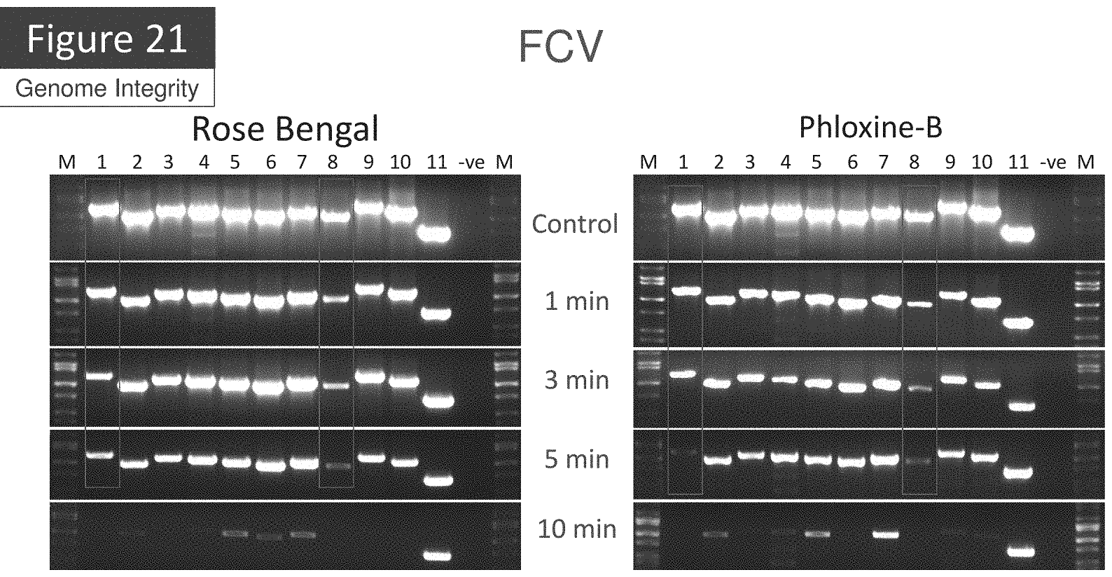

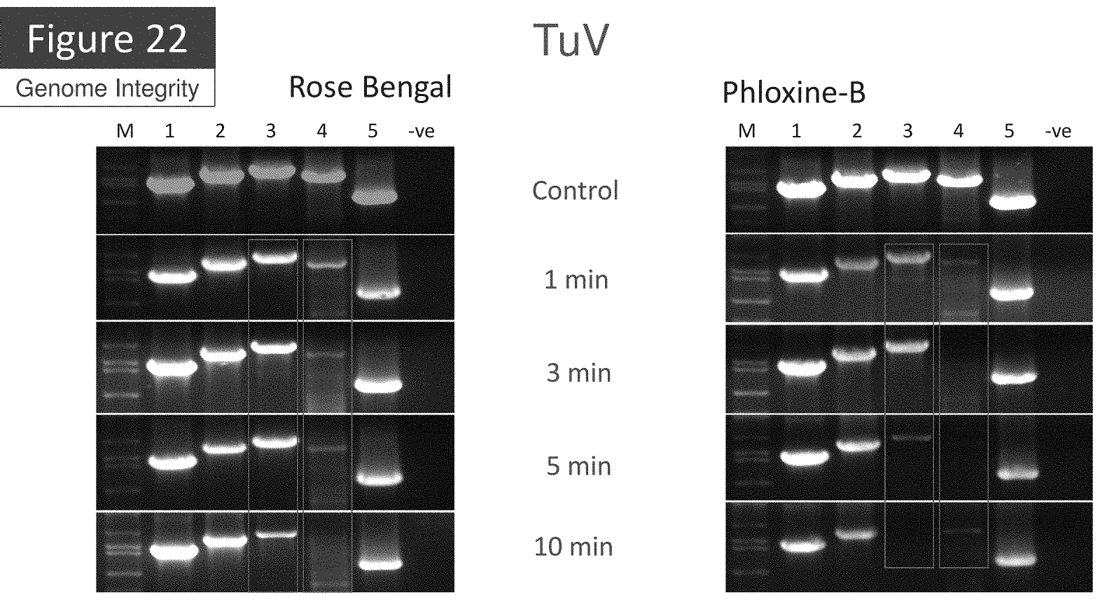

As shown in FIGS. 8-17, photodynamic treatment can inactivate HuNoV surrogates in water and on surfaces. Blue LED light was more efficient than white fluorescent and halogen light. Under the conditions tested, chlorophyllin had low efficacy against FCV while RB and Ph-B had high efficacy against FCV and TuV. The efficacy of RB and Ph-B was dependent on exposure time and concentration. A 4 log reduction in FCV was observed after a 3 minute exposure to 25 μM RB or 50 μM Ph-B and a 4 log reduction in TuV was observed after a 10 minute exposure to 50 μM RB or 5 minute exposure to 50 μM Ph-B. ROS of PDT inactivate FCV and TuV mainly by its oxidative impact on the viral capsid (oxidation and/or disintegration) followed by disintegrating the viral genome (FIGS. 19 and 21-22).

Thus, a single exposure to an aqueous solution or a surface having virus of PDT and a PS can reduce viral load, e.g., detectable virus including viable virus, by at least 4 logs.

The method may be employed to inactivate any virus, enveloped or non-enveloped. Any photosensitizer that allows for inactivation of viruses when exposed to blue light may be employed in the methods. In one embodiment, the photosensitizer may be RB or a derivative thereof, e.g., derivatives disclosed in Sugita et al., Bioconjugate Chem., 18:866 (2007), Paczkowski et al., Tetrahedron, 43:4579 (1987), Paczkowski et al. J. Free Radicals Biol. Med., 1:341 (1985), Paczkowska et al., Photche. Photobiol., 42:603 (1985), the disclosures of which are incorporated by reference herein. Other red dyes that may be employed in the methods include but are not limited to Ponceau 4R, carmoisine, erythrosine, azorubine, Allura Red AC, orange B or FD&C red no. 32. Also, other LED lights with different color and different wavelengths can be employed to enhance the efficacy of Chlorophyllin which have the advantage that it is natural compound and edible.

### Exemplary Embodiments

This disclosure provides methods and systems, e.g., sources that emit blue light and photosensitizers, that inhibit viral viability, or inactivate viral infectivity, on a surface, e.g., used to prepare food or other inanimate surfaces found in a house, boats such as cruise ships, and restaurants, or on a highly-touched surfaces in hospitals, vehicles, boats or grocery stores, and other settings that come into contact with human skin, such as hand rails, door knobs, surfaces of a door or in an elevator, gym equipment, and the like, or food product or in an aqueous solution. The method includes exposing the surface, food product or aqueous solution to a photosensitizer and selected bands or a wavelength of light for a period of time, so as to inhibit viruses. Thus, the method the use of one or more light sources that emit radiation at one or a plurality of wavelengths (i.e., in a particular spectral region or “band” which does not represent the spectrum emitted by white light). The exposure is for a period of time, e.g., less than one minute up to 30 minutes, or more.

In the methods, one or more light sources may be employed. Thus, one light source may be used to emit one or a band of wavelengths. The method can alternatively employ two, or more than two, light sources, each to emit the same of the different bands or wavelengths. The light source can comprise at least one incandescent lamp, a fluorescent lamp, a high sodium vapor lamp or a light emitting diode lamp, although the invention is not limited to those sources, as well as combinations of light sources, and, if necessary, a means to filter the spectrum of wavelengths which are emitted. In one embodiment, a single light source is employed to provide the desired band or wavelength of light. In one embodiment, one light source is employed that comprises an incandescent lamp with a blue filter. In another embodiment, more than one light source is employed.

The amount of light emitted may, in one embodiment, reduce viral load by 10² to 10¹⁰, e.g., reduce load by at least two to three logs, such as reduce by 10³ to 10⁶, 10⁴ to 10⁸, or 10⁵ to 10⁷ IU, PFU or TCID50, or reduce viral load from, for example, 10⁶ to 10³, 10⁸ to 10⁴, or 10⁷ to 10⁵ TCID₅₀, PFU or IU. The amount of light emitted may, in one embodiment, reduce bacterial load by 10² to 10¹⁰, e.g., reduce load by at least two to three logs, such as reduce by 10³ to 10⁶, 10⁴ to 10⁸, or 10⁵ to 10⁷ CFU or reduce CFU from 10⁶ to 10³, 10⁸ to 10⁴, 10⁷ to 10⁵, 10⁶ to 10², 10⁸ to 10¹, or 10⁷ to 10².

In one embodiment, a light source circuit includes one or more light-emitting diodes (LEDs) each functioning as a light source. In one embodiment, each of the one or more LEDs emits a light having one or more specified wavelengths or ranges of wavelength. In another embodiment, each of the one or more LEDs is provided with an optical filter to produce the light having a specified wavelength or range of wavelength. In one embodiment, a light source circuit includes a plurality of LEDs, connected in parallel and powered by a voltage source V. Each of the LEDs may be current-limited using a resistor R and is turned on or off using a switch S. In one embodiment, LED1 through LEDN are of the same type and emit a light with a single wavelength. In another embodiment, LED1 through LEDN include two or more types of LEDs emitting lights having substantially different wavelengths. In one embodiment, the light emits 100 mcd to 1000 mcd, e.g., 600 to 800 mcd, or any individual integer or range therein. In one embodiment, the light emits about 0.01 to about 5 mW/cm², e.g., at a surface. In one embodiment, the light emits about 200 to 800 lumens/meter, e.g., 400 to 500 lumens/meter, or any individual integer or range therein, e.g., at a surface. In one embodiment, the light emits about 7,000 to 11,000 mW/meter, e.g., 8,000 to 11,000 or 9,000 to 10,000 mW/meter, or any individual integer or range therein, e.g., at a surface. In one embodiment, the light emits about 5 to 20 lumens/module, e.g., 10 to 15 or 20 to 12 lumens/module, or any individual value or range therein, e.g., at a surface.

In one embodiment, a method to decrease viral load on a surface, food product or in an aqueous solution is provided. The method includes providing a surface, food product or an aqueous solution suspected of having virus; and contacting the surface, food product or solution with an amount of a photosensitizer and an amount of blue light for a period of time effective to decrease viral load on the surface or the food product or in the aqueous solution. In one embodiment, the surface is contacted with the photosensitizer and then the blue light. In one embodiment, the aqueous solution is contacted with the photosensitizer and the blue light. In one embodiment, the photosensitizer is a fluorescein. In one embodiment, the fluorescein is halogenated. In one embodiment, the photosensitizer is halogenated. In one embodiment, the photosensitizer has 1 to 4 Cl, Fl, Br or I. In one embodiment, the photosensitizer has 1 to 4 Cl and 1 to 4 Br. In one embodiment, the photosensitizer has 1 to 4 Cl and 1 to 4 I. In one embodiment, the photosensitizer is a red dye. In one embodiment, the photosensitizer is Rose Bengal, Phloxine B, Methylene Blue, Flavin mononucleotide, Curcumin, or a salt thereof. In one embodiment, the light has a wavelength of about 400 nm to 750 nm or any visible wavelength of light. In one embodiment, the light has a wavelength of about 460 nm to 480 nm. In one embodiment, the light has a wavelength of about 450 nm to 470 nm. In one embodiment, the virus is an enveloped virus. In one embodiment, the virus is a non-enveloped virus. In one embodiment, the virus is a single or doubled stranded DNA virus. In one embodiment, the virus is a single or doubled stranded RNA virus. In one embodiment, the virus is a parvovirus, poxvirus, herpesvirus, hepatitis virus, enterovirus, norovirus, coronavirus, or any human virus, non-human animal virus or plant virus. In one embodiment, the viral load is decreased by at least 2 logs. In one embodiment, the viral load is decreased by at least 6 logs. In one embodiment, the food product is contacted with the light and the photosensitizer. In one embodiment, the food product is fresh produce.] or berries In one embodiment, the food product is shellfish. In one embodiment, the surface is a steel, granite, marble, wood, rubber, ceramic, plastic, or glass surface. In one embodiment, the aqueous solution has a pH between 5 and 9. In one embodiment, the aqueous solution has a pH between 6 and 8.

The invention will be described by the following non-limiting examples.

### Example 1

Shellfish and fresh produce are the most frequent foods implicated in human norovirus (HuNoV) outbreaks in the US. Decontamination of virus-contaminated waters used in depuration of oysters, clams or mussels, and in post-harvest washing of fresh produce, is a mitigation strategy for this human biothreat risk. Photodynamic treatment (PDT), a non-thermal technology, is a suggested virucidal intervention process. Although the PDT has been studied extensively for bacterial inactivation, studies on its use for viral inactivation in food and water are scarce. Inactivation of >4 log TCID50 of two HuNoV surrogates (feline calicivirus (FCV) and Tulane virus (TuV)) in water was observed upon exposure to LED-blue light for 3 to 10 min using Rose Bengal (RB) or Phloxine-B (Ph-B) as photosensitizers. The virucidal effect of PDT may depend on the type of treated virus, the light source, the type and concentration of the photosensitizer (PS), and the operational parameters such as the distance between the light source and treated object and/or exposure time.

The integrity of capsid proteins and genomes of PDT-treated FCV and TuV as compared to untreated viruses (control) was determined. Electron microscopy imaging and several molecular techniques such as RT-PCR and RNase-coupled RT-qPCR were used to determine efficacy.

The results indicated that the reactive oxygen species (ROS) of PDT inactivates both FCV and TuV mainly by disintegrating a majority of the viral capsid. However, a small fraction of the viral particles was inactivated while retaining intact capsids, which indicates that the oxidative impact of the PDT's ROS on some functional peptides or amino acids in the viral capsid is another possible mode of action. In addition, we observed increasing disintegration of the viral genome over time of exposure, which appears to be due to uncoating of the genome after the damage occurs in the viral capsids.

Understanding the virucidal mode-of-action may allow for optimizing PDT/PS technology for mitigating the risk of food borne viruses, e.g., HuNoV, hepatitis virus or other enteric viruses, contamination in waters used in shellfish farms and post-harvest stations of fresh produce.

### Example 2

Romaine lettuce leaves were cut into small squares (2×2 cm) then disinfected using 300 ppm sodium hypochlorite followed by rinsing three times with sterilized distilled water. Sterile stainless steel discs were also used as model for surface decontamination. Each sample was spiked with 50 μL of a virus suspension, e.g., transmissible gastroenteritis virus (TGEV) as a surrogate to coronavirus (COVID-19 or SARS-Cov-2) and FCV as a surrogate for HuNoV, then dried for 30 minutes. 100 μL of a 50 μM Phloxin-B solution was then added to on the spiked leaf surface. The samples were exposed at 1 cm distance to the LED light for 1, 3, 5, and 10 minutes, after which virus was recovered in 500 uL of virus recovery buffer followed by vortex for 0.5 to 1 minute. The titer of surviving viable virus was measured in swine testicle (ST) cell monolayers using the 50% end-point technique and calculated by the Karber's method.

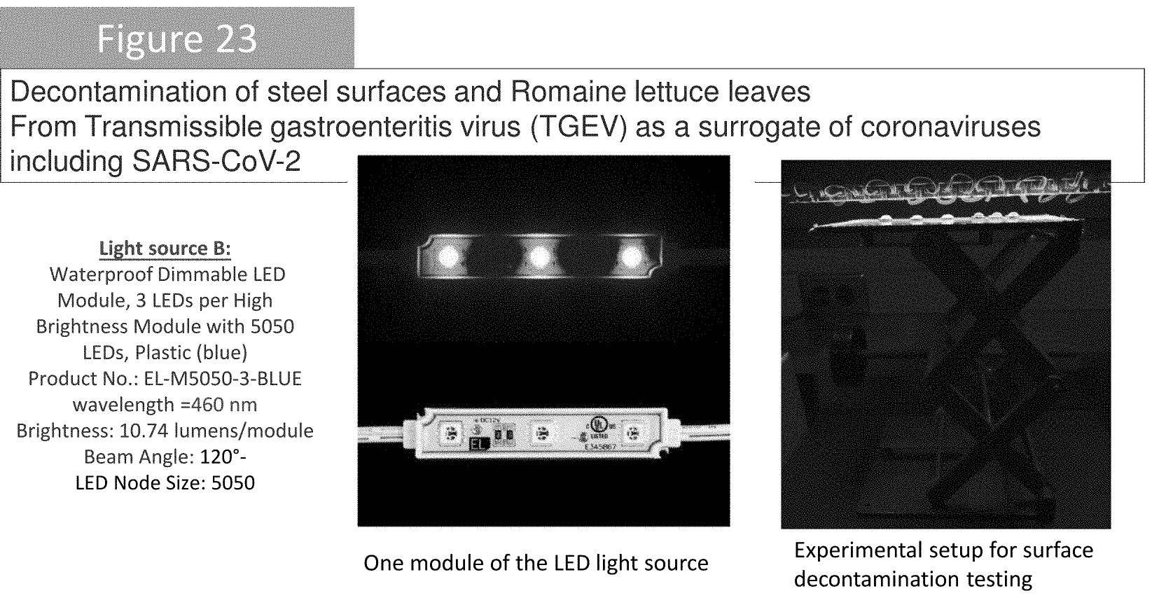

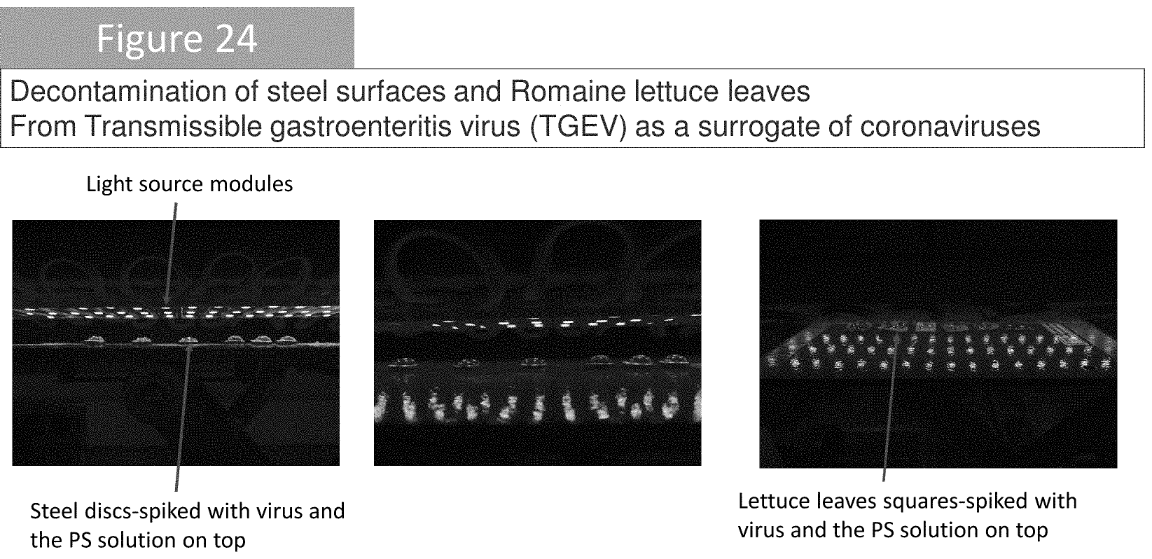

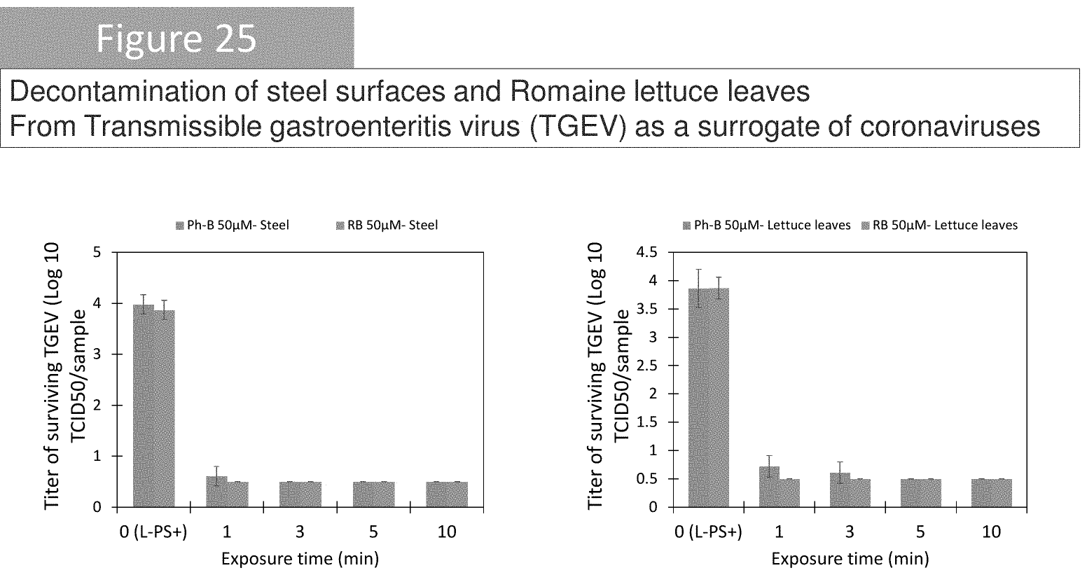

As shown in FIGS. 23-25, the use of PS and PDT (at 1 cm from the surface and 50 uM PS) allowed for very effective decontamination of TGEV on both stainless steel and romaine lettuce leaves>99.9% in only 1 min exposure to blue LED light. Compared to non-enveloped viruses, TGEV, and thus coronaviruses (both enveloped viruses), are more susceptible to PS and PDT. Thus, PDT and PS may be very effective for commercial applications for surface decontamination with regards to COVID-19.

### Example 3

Romaine lettuce leaves are cut into small squares (2×2 cm) and then are disinfected using 300 ppm sodium hypochlorite followed by rinsing three times with sterilized distilled water. Sterile stainless-steel discs are also used as a model for surface decontamination. Each sample is spiked with 50 μL of a Gram positive or Gram negative bacterial suspension, e.g., E. coli, Salmonella enterica, Staphylococcus aureus, or Bacillus subtilis, then dried for 30 minutes. 100 μL of a 50 μM Phloxin-B solution is then added to each spiked leaf or stainless-steel surface. The samples are exposed at a 1 cm distance to the blue LED light source for 1, 3, 5, and 10 minutes. After each exposure time any surviving bacterial cells are recovered in 500 μL of recovery buffer followed by vortexing for 0.5 to 1 minute. The surviving bacterial cells are enumerated using Agar plate count technique. A ≥3 log reduction in the bacterial cell counts is observed.

### Example 4

Whole romaine lettuce leaves are washed and disinfected using 300 ppm sodium hypochlorite followed by rinsing three times with sterilized distilled water. Each whole leaf is spiked with 500 μL of a virus suspension, e.g., transmissible gastroenteritis virus (TGEV) as a surrogate to coronavirus (COVID-19 or SARS-Cov-2) and FCV as a surrogate for HuNoV, then dried for 30 minutes. One sample at a time is placed in a 300 mL capacity container.

Several 50 μM stocks of Phloxin-B solution are prepared (500 μL each). Each stock is pre-exposed to the blue LED light for specific times (e.g., 1, 2, 3, 5, and 10 min). Immediately after light exposure, 50 μL of the tested virus (FCV, TuV, and TGEV) is added to the light-preexposed Ph-B solution for about 1 to 5 min contact times before titrating the surviving virus using a cell culture assay. A >3 log reduction in the virus titer is observed. The results may show that the light-preexposed photosensitizer solution is an environmentally friendly surface disinfectant and a washing/decontamination step for use in produce post-harvest stations and in food processing plants.

### REFERENCES

Marsh et al. (2018) Food Safety, 6:58.
Bartsch et al. (2016) PLoS One, 11:e0151219.
Scharff (2012) J. Food Prot. 75:123.

It is to be understood that the above detailed description is intended to be illustrative, and not restrictive. Other embodiments will be apparent to those of skill in the art upon reading and understanding the above description. The scope of the invention should, therefore, be determined with reference to the appended claims, along with the full scope of equivalents to which such claims are entitled.

What is claimed is:

1. A method to decrease microbial load on a surface, food product or in an aqueous solution, comprising:
providing a surface, food product or an aqueous solution suspected of having microbial contamination; and
exposing the surface, food product or solution to an amount of a photosensitizer and an amount of blue light for a period of time effective to decrease the amount of viable microbe on the surface or the food product or in the aqueous solution.

2. The method of claim 1 wherein the photosensitizer is a fluorescein.

3. The method of claim 2 wherein the fluorescein is halogenated.

4. The method of claim 1 wherein the photosensitizer is halogenated.

5. The method of claim 4 wherein the photosensitizer has 1 to 4 Cl, Fl, Br or I.

6. The method of claim 1 wherein the photosensitizer is a red dye.

7. The method of claim 1 wherein the photosensitizer is Rose Bengal, Phloxine B, chlorophyllin, or a salt thereof.

8. The method of claim 1 wherein the light has a wavelength of about 450 nm to 490 nm.

9. The method of claim 1 wherein the light has a wavelength of about 450 nm to 470 nm.

10. The method of claim 1 wherein the light has a wavelength of about 460 nm to 475 nm.

11. The method of claim 1 wherein the light has a wavelength of about 460 nm to 480 nm.

12. The method of claim 1 wherein the exposure decreases viral load by at least 4 logs.

13. The method of claim 1 wherein the exposure decreases viral load by at least 2 logs.

14. The method of claim 1 wherein the exposure decreases bacterial load iby at least 3 logs.

15. The method of claim 1 wherein the food product is exposed to the light and the photosensitizer.

16. The method of claim 15 wherein the food product is produce.

17. The method of claim 15 wherein the food product is fruit.

18. The method of claim 15 wherein the food product is shellfish.

19. The method of claim 1 wherein the surface is a steel, granite, marble, wood, rubber, ceramic, plastic, fabric, or glass surface.

20. The method of claim 1 wherein the aqueous solution has a pH between 6 and 8.
